(19) United States
(12) Patent Application Publication
(10) Pub. No.: US 2026/0151095 A1
(43) Pub. Date: Jun. 4, 2026

(54) POSITIONING DEVICE FOR WEIGHT BEARING MERCHANT VIEWS OF THE KNEE

(71) Applicant: New York Society for the Relief of the Ruptured and Crippled, maintaining the Hospital for Special, New York, NY (US)

(72) Inventors: Fernando J. Quevedo-Gonzalez, Greenwich, CT (US); David j. Mayman, New York, NY (US); Ralph Lopez, Glen Cove, NY (US); Yolanda Wojcik, Clark, NJ (US)

(73) Assignee: New York Society for the Relief of the Ruptured and Crippled, maintaining the Hospital for Special, New York, NY (US)

(21) Appl. No.: 19/123,237

(22) PCT Filed: Oct. 27, 2023
(86) PCT No.: PCT/US2023/078071

§ 371 Date: Apr. 22, 2025

Related U.S. Application Data
(60) Provisional application No. 63/419,814, filed on Oct. 27, 2022.

(51) Int. Cl. A61B 6/04; A61B 6/50

(52) U.S. Cl. CPC A61B 6/0407; A61B 6/505

(57) ABSTRACT

A positioning device for generating merchant views of the knee joint is disclosed. The positioning device includes a frame. The positioning device also includes a femoral positioning plate coupled to the frame and configured to position a femur of the patient in a predefined position. Additionally, the positioning device includes a tibial positioning plate coupled to the frame and configured to position a tibia of the patient in about 45 degrees of flexion.

### BACKGROUND

The knee includes tibiofemoral and patellofemoral joints. The patellofemoral joint includes the patella and the trochlear groove of the femur. Due to osteoarthritis or traumatic events, the patellofemoral joint may become damaged requiring surgical intervention.

Conventional merchant radiographic views allow surgeons to assess the status of the patellofemoral joint. This radiographic view creates a horizon view of the patellofemoral joint that shows the patellofemoral joint space, allowing a physician to measure relevant radiographic parameters, like patellar tilt, patellar subluxation, or patellofemoral contact. To acquire this standardized radiographic view, patients lay supine, while their knees are positioned in 45° flexion. During acquisition of this supine merchant view, the extensor mechanism of the knee (i.e., the quadriceps muscle) remains relaxed.

However, during function of the knee, the extensor mechanism activates to allow knee motion which affects the position of the patella relative to the trochlear groove. Therefore, the position of the patella within the trochlear groove in supine merchant views may differ from that during daily activities, therefore hampering appropriate functional evaluation of the patella.

As such, a need remains to allow evaluation of the patella in its functional position. Such a need is satisfied by the positioning device of the present disclosure.

### BRIEF SUMMARY OF THE DISCLOSURE

The subject disclosure provides an apparatus and method to evaluate the patella in its functional position by acquiring a functional radiographic view equivalent to the standard supine merchant view of the patellofemoral joint where the extensor mechanism of the knee is active. Activation of the extensor mechanism of the knee can be achieved with a weight-bearing merchant view with the patient in a squatting position. The apparatus includes a positioning device that ensures approximately 45° knee flexion and approximately 30° between the femur and the X-ray beam.

The positioning device is configured to consistently achieve a repeatable position of patients to obtain a weight-bearing merchant view of the patellofemoral joint reproducing that of a supine merchant view. The positioning device helps position the shin (i.e., tibia) and thigh (i.e., femur) of the patient such that the knee flexion is approximately 45° and the thigh is at approximately 30° with respect to the X-ray beam.

The patient can be standing in a squatting position, so that their knee extensor mechanism (i.e., quadriceps muscle) is active. The X-ray source can be above the patient and the X-ray beam can be directed vertically towards the ground. The radiographic cassette can be placed perpendicular to the X-ray beam (i.e., horizontally) below the patient's knee and above the feet, on a support platform that is part of the device's frame. The positioning device can include a frame comprising interconnected bars, arranged to provide sufficient stability to the device, to support the different positioning elements, to support the radiographic cassette, and to support the positioning elements. The frame can be made of plates or any other structural element.

The device can include at least a positioning plate for the tibia and a positioning plate for the femur. The device can provide an unobstructed view of the patellofemoral joint. This can be achieved by either radiolucent elements and/or by avoiding placing radiopaque elements in locations that can obscure the knee in the radiographic image. The device frame can have an opening such that the patient can access the positioning plates. The femoral and tibial positioning plates can be placed in front of the patient's legs or behind the patient's legs. The femoral and tibial positioning plates can be placed to leave sufficient clearance of the patellofemoral joint and ensure that they do not affect the position of the patella within the trochlear groove. The femoral positioning plate can form an angle of approximately 30° with the vertical, while the tibial positioning plate can form an angle of approximately 15° with the vertical. The tibial and femoral positioning plates can be fixed elements. One or both positioning plates can also be mobile elements, for example, using linear bearings or any other suitable elements that allow motion. The femoral and tibial positioning plates can move independently or move as a unit. In one configuration, the tibial positioning plate can be fixed to the frame of the device, while the femoral positioning plate is mobile. The femoral positioning plate can move along an axis oriented approximately 15° with respect to the vertical, parallel to the tibial plate, such that the distance between the femoral plate and the knee remains constant for all heights of patients.

The positioning device advantageously provides functional views of the patellofemoral joint. In other words, the positioning device functions to repeatably acquire weight bearing merchant views of the knee.

In accordance with an exemplary embodiment, the subject disclosure provides a positioning device for generating merchant views of the knee joint, the positioning device comprising: a frame; a tibial positioning plate coupled to the frame and having a major face at about 15° relative to a vertical plane; and a femoral positioning plate coupled to the frame and above the tibial positioning plate, the femoral positioning plate having a major face at an angle relative to a plane defined by the tibial positioning plate. The femoral positioning plate is disposed at an angle of approximately 45° relative to the plane defined by the tibial positioning plate. The femoral positioning plate is disposed at an angle of approximately 30° relative to a longitudinal axis of the positioning device. One or more of the tibial positioning plate and the femoral positioning plate are movable relative to one another. The tibial positioning plate is fixedly coupled to the frame and the femoral positioning plate is movable relative to the frame. The femoral positioning plate is moveable along an axis oriented about 15 degrees with respect to a longitudinal axis of the positioning device. The femoral positioning plate is moveable along an axis parallel to a major face of the tibial positioning plate. The frame includes one or more handles configured for patient engagement. The handles are above the femoral positioning plate. The frame includes a pair of rails each having a handle assembly movable along the rail. The femoral positioning place is spaced from the tibial positioning plate. The femoral positioning plate is spaced from the tibial positioning plate sufficiently to allow a knee joint therebetween. The femoral positioning plate is spaced from the tibial positioning plate sufficiently to allow a knee joint to be positioned anteriorly of the femoral and tibial positioning plates. The femoral positioning plate is sized sufficiently to receive a posterior side of a patient's legs.

In accordance with another exemplary embodiment, the subject disclosure provides an x-ray system comprising: an x-ray machine configured to output an x-ray beam at an x-ray beam angle of about 0° relative to a vertical axis; and a positioning device that includes: a frame having a longitudinal axis substantially parallel to the x-ray beam angle, a femoral positioning plate coupled to the frame and disposed at an angle of approximately 30° relative to the vertical axis. The x-ray system further comprises a tibial positioning plate coupled to the frame and disposed at an angle of approximately 45° relative to a plane defined by a major face of the femoral positioning plate. One or more of the tibial positioning plate and the femoral positioning plate are movable relative to one another. The femoral positioning plate is above the tibial positioning plate. The positioning system includes handles positioned above the tibial positioning plate.

In accordance with another exemplary embodiment, the subject disclosure provides a method of evaluating a patella of a patient in its functional position, the method comprising: positioning a patient on a positioning device as discussed above; and obtaining a radiograph of the patient's patella while positioned on the positioning device in a weight bearing position.

### BRIEF DESCRIPTION OF THE SEVERAL VIEWS OF THE DRAWINGS

The foregoing summary, as well as the following detailed description of the exemplary embodiments, will be better understood when read in conjunction with the appended drawings. For the purpose of illustration, there are shown in the drawings exemplary embodiments of the subject disclosure. It should be understood, however, that the subject disclosure is not limited to the precise arrangements and instrumentalities shown.

In the drawings:
FIG. 1 is supine merchant view of a patient's patella according to the prior art;

FIG. 2 is a schematic view of a patient arranged for the supine merchant view of FIG. 1;

FIGS. 3A-B show the supine merchant view of FIG. 1 compared to a weight bearing merchant view of the patient's patella according to the present disclosure;

FIG. 4 is a schematic perspective view of a positioning device according to the present disclosure;

FIG. 5 is schematic side view of the positioning device of FIG. 4;

FIG. 6 is a schematic front view of the positioning device of FIG. 4; and

FIG. 7 is a schematic perspective view of a positioning device according to another aspect of the present disclosure.

### DETAILED DESCRIPTION OF THE INVENTION

Reference will now be made in detail to the various examples of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages, and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

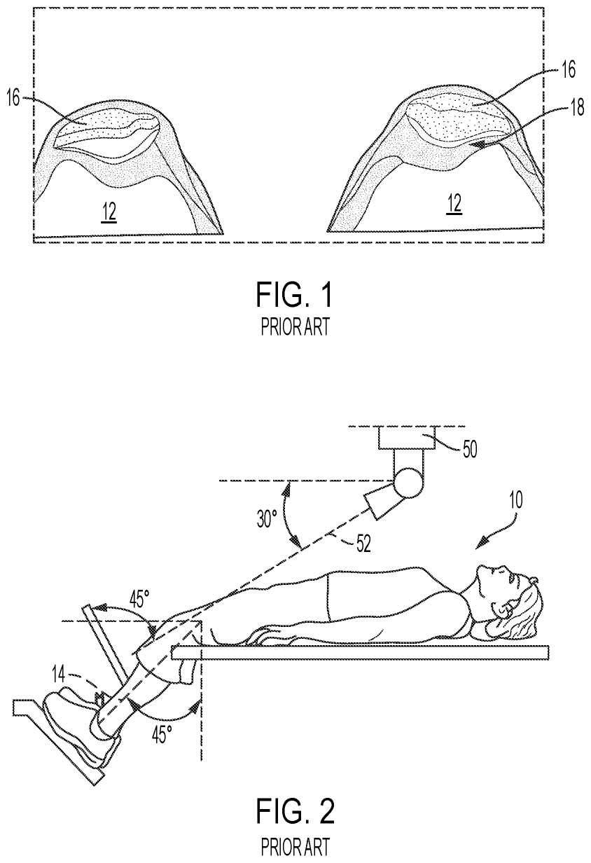

Referring now to FIGS. 1 and 2, the knee of a patient 10 is formed by a plurality of joints including the tibiofemoral and patellofemoral joints which join a femur 12 and a tibia 14. The patellofemoral joint includes the patella 16 and the trochlear groove 18 of the femur.

Conventional merchant radiographic views, such as the radiographic merchant view shown in FIG. 1, allow physicians to assess the status of the patellofemoral joint. This radiographic view creates a horizon view of the patellofemoral joint that shows the joint space, allowing to measure relevant radiographic parameters, like patellar tilt, patellar subluxation, or patellofemoral contact. To acquire this standardized radiographic view, conventional technique is to have a patient lay supine, while their knees are positioned in 45° flexion, as shown in FIG. 2. During acquisition of this supine merchant view, the extensor mechanism of the knee (i.e., the quadriceps muscle) remains relaxed.

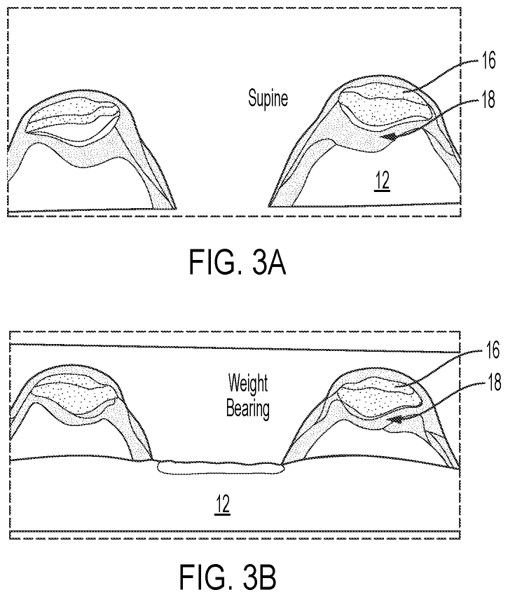

However, during function of the knee, the extensor mechanism activates to allow knee motion which affects the position of the patella 16 relative to the trochlear groove 18. Therefore, the position of the patella 16 within the trochlear groove 18 in supine merchant views may differ from that during daily activities, therefore hampering appropriate functional evaluation of the patella. This is illustrated in FIGS. 3A and 3B which show a supine merchant view in FIG. 3A and a weight-bearing merchant view in FIG. 3B.

To assist in achieving this repeatable weight-bearing merchant view as shown in FIG. 3B, a positioning device 20 for weight bearing merchant views of the knee is provided. In other words, the positioning device 20 as described herein functions to repeatably acquire weight bearing merchant views of the knee. The positioning device 20 includes a frame 22, a femoral positioning plate 24, and a tibial positioning plate 26.

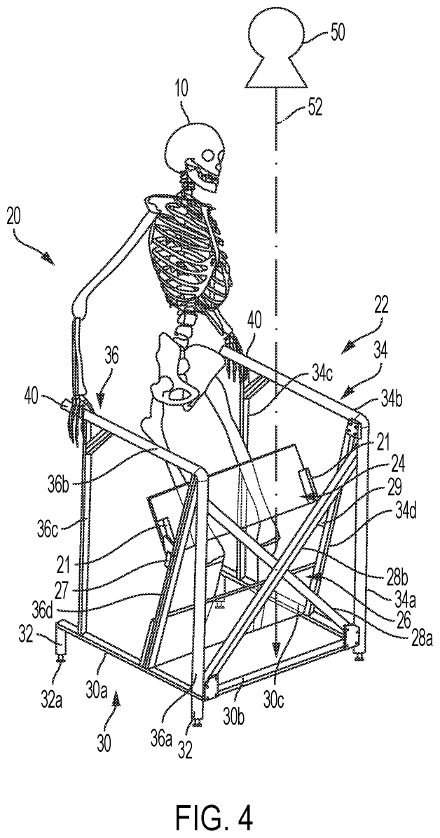

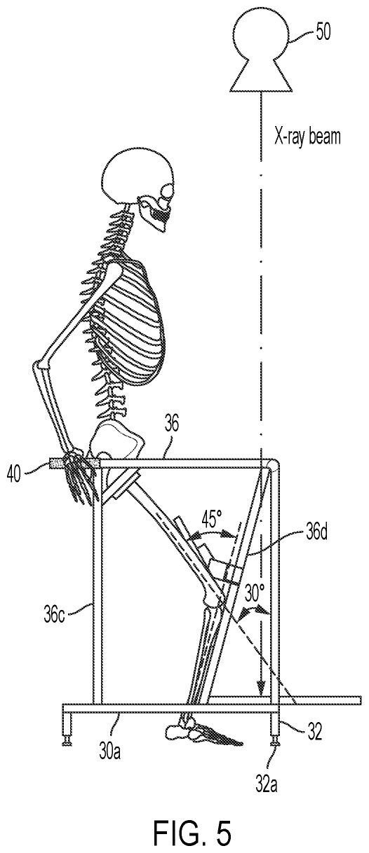

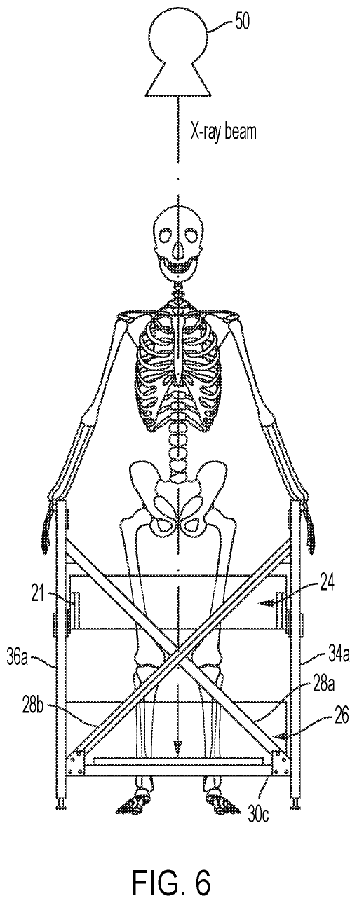

The positioning device 20 is configured, for example, as shown in FIGS. 4-6 in accordance with an exemplary embodiment of the subject disclosure. The frame 22 includes interconnected rails or beams, arranged to provide sufficient stability to the device. FIGS. 4-6 illustrate an exemplary frame configuration, but the frame can be alternatively configured so long as it meets the required intended use. For example, the frame 22 may be configured to include an opening such that the patient 10 can access the femoral positioning plate 24 and the tibial positioning plate 26.

The frame 22 includes a series of interconnected rails. The rails 28 are generally cylindrical, however, various other configurations have been contemplated including planar bars or a combination thereof and the like. The rails 28 may be secured to one another through known methods such as welding and the like. In some examples, the frame 22 is a single piece construct such that movement of one portion of the frame 22 induces movement in the same direction to the remaining portions of the frame 22. However, the frame may be configured to be modular so as to allow interconnection and replacement of parts, such as through fasteners, brackets, and the like. The frame may also be configured to have some or all portions of the frame 22 be movable relative to other portions, if desired, so as to adjust for height, depth and width to accommodate patients of differing size.

The frame 22 is configured as a generally rectangular frame. The frame includes a substantially rectangular base 30 formed by rails 30a, 30b, 30c. The substantially rectangular base includes a front rail 30b connecting side rails 30a and 30c, but does not include a back rail thereby defining an opening such that a user may easily be positioned within the base 30 or enter the positioning device. In alternative aspects, the base of the frame can be any suitable shape such as, for example, substantially square, substantially a pentagon, substantially circular, substantially ovoid, etc. In aspects of the disclosure, rails 30a and 30c are an optional feature. In some examples, the frame 22 may include one or more floor engaging portion 32 extending downward from one or more corners of the base 30 and configured to engage the floor thereby raising the base 30 off the floor. The floor engaging portions 32 may be any length and or may be adjustable such that the positioning device 20 may accommodate patients of various heights and to adjust for level. For example, the floor engaging portion may include an adjustment knob 32a, such as a threaded knob. In aspects of the disclosure, the floor engaging portion can include wheels.

The frame 22 also includes a left arm rail assembly 34 and a right arm rail assembly 36. Each of the left and right arm rail assemblies extend upwardly from the base 30. That is, the left arm rail assembly 34 extends from rail 30c and the right arm rail assembly 36 extends from rail 30a.

The left arm rail assembly 34 includes a front vertical rail 34a, a top horizontal rail 34b, a rear vertical rail 34c, and a cross-bar rail 34d. The cross-bar rail 34d extends from rail 30c to about a corner defined by rails 34a and 34b, at an angle of about 30 degrees relative to a longitudinal angle of the front vertical rail 34 a.

The right arm rail assembly 36 includes a front vertical rail 36a, a top horizontal rail 36b, a rear vertical rail 36c, and a cross-bar rail 36d. The cross-bar rail 36d extends from rail 30a to about a corner defined by rails 36a and 36b, at an angle of about 30 degrees relative to a longitudinal angle of the front vertical rail 36 a.

Each of the left and right rail assemblies include a handle 40 that can be easily grasped by a user. The handles 40 can be one and the same as the horizontal rails 34b, 36b, or they can be additional handles added to the horizontal rails 34b, 36b. Each handle is configured for patient engagement.

As the rails 34b, 36b serve as supports or patient engaging portions for the user, the rails 34b, 36b are aligned so as to be horizontal to each other (e.g., aligned to be on the same horizontal plane or at the same height relative to the floor). The patient engaging portions extends approximately the same length as the base 30, however, various other configurations have been contemplated.

The frame 22 may additionally include cross-bars 28a, 28b for additional stability.

The frame 22 may also include additional elements including but not limited to plate support(s) 21 for supporting the femoral positioning plate 24. Additional supports may be incorporated to support the tibial positioning plate 26, a radiographic cassette, and/or separate handles or other engagement mechanisms.

The femoral positioning plate 24 is coupled to the frame 22 and configured to positioning or engage a femur 12 of the patient 10 in a defined weight bearing position. The femoral positioning plate is positioned above the tibial positioning plate 26. In some examples, the femoral positioning plate 24 includes a planar upper surface (i.e., a major face) and a planar lower surface coupled by corresponding planar side surfaces. In the example shown, the femoral positioning plate 24 in generally rectangular having a length which extends between the left and right arm rail assemblies of the frame 22 and a width which is shorter than the length but of sufficient width to provide positioning to the femur 12 of the patient. Additionally, the femoral positioning plate 24 may be of any thickness configured to provide positioning to the femur 12 of the patient or additionally include other features e.g., padding.

In the example shown, the femoral positioning plate 24 is disposed at an angle of approximately 30 degrees relative to a longitudinal axis of the positioning device 20, or a vertical axis or vertical plane. However, other angles have been contemplated including but not limited to approximately 5, 10, 15, 20, 25, 35, 40, 45, and 50 degrees from the longitudinal axis of the positioning device 20 or a vertical plane. In other words, the major face of the femoral positioning plate is at an angle relative to a plane defined by the tibial positioning plate. For example, the femoral positioning plate is disposed at an angle of approximately 45 degrees relative to the plane defined by the tibial positioning plate.

The femoral positioning plate 24 is spaced from the tibial positioning plate 26. That is, the femoral positioning plate is spaced from the tibial positioning plate sufficiently to allow a knee joint therebetween or sufficiently to allow a knee joint to be positioned anteriorly of the femoral and tibial positioning plates.

The positioning device 20 also includes the tibial positioning plate 26 coupled to the frame 22 and configured to positioning the tibia 14 of a patient in a defined weight bearing position. In some examples, the tibial positioning plate 26 includes a planar upper surface (i.e., a major face) and a planar lower surface coupled by corresponding planar side surfaces. In the example shown, the tibial positioning plate 26 in generally rectangular having a length which extends between the left and right arm rail assemblies of the frame 22 and a width which is shorter than the length but of sufficient width to provide positioning to the tibia 14 of the patient. Additionally, the tibial positioning plate 26 may be of any thickness configured to provide positioning to the tibia 14 of the patient, or additionally include other features e.g., padding.

In the example shown, the tibial positioning plate 26 is disposed at an angle of approximately 15° from a longitudinal axis of the positioning device 20 or a vertical plane. However, other angles have been contemplated including but not limited to approximately 4, 5, 6, 8, 10, 12, 14, 16, 18, 20, 22, 24, 26, 28, 30, 35, 40 degrees from the longitudinal axis of the positioning device 20 or a vertical plane.

In some examples, the femoral positioning plate 24 and the tibial positioning plate 26 have similar dimensions. In other examples, the femoral positioning plate 24 has a larger width and/or thickness than the tibial positioning plate 26 to accommodate various sizes of the patient's femur.

In some examples, the femoral positioning plate 24 and the tibial positioning plate 26 are fixed elements such that neither the femoral positioning plate 24 or the tibial positioning plate 26 are movable with respect to each other or with respect to the frame 22. In other examples, one or more of the femoral positioning plate 24 and the tibial positioning plate 26 are movable elements. In some examples, one or more of the femoral positioning plate 24 and the tibial positioning plate 26 are movable relative to each other and/or relative to the frame 22. In some examples, the femoral positioning plate 24 and the tibial positioning plate 26 are movable as a unit such that movement of the femoral positioning plate 24 or the tibial positioning plate 26 correlates with movement of the other.

To facilitate this movement, one or more of the femoral positioning plate 24 or the tibial positioning plate 26 may include pivots 27 or bearings 29, such as linear bearings, or other components which facilitate movement. In one example, the tibial positioning plate 26 is fixed to the frame 22 while the femoral positioning plate 24 is movable. In this example, the femoral positioning plate 24 may move along an axis orientated approximately 15° from the longitudinal axis and parallel to the tibial positioning plate 26 such that the distance between the femoral positioning plate 24 and the knee remains constant for patients of varying heights. However, various other configurations have been contemplated including the femoral positioning plate 24 moving along an axis orientated 5-35 degrees from the longitudinal axis. In other examples, the femoral positioning plate 24 is movable along an axis which is parallel to the planar upper surface or the planar lower surface of the tibial positioning plate 26. However, various other configurations have been contemplated.

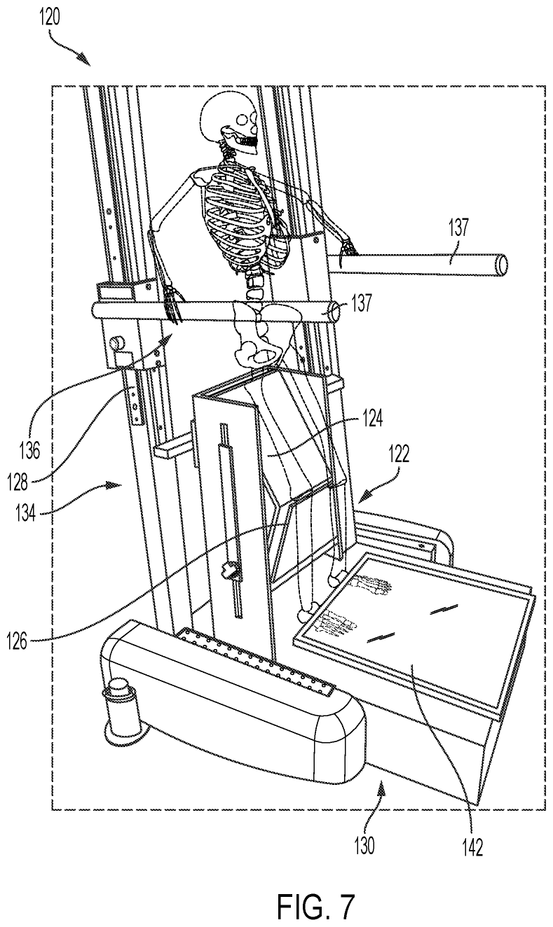

FIG. 7 illustrated another exemplary embodiment of a positioning device 120 in accordance with the subject disclosure. The positioning device 120 may be similar to the positioning device 20 described above including but not limited to the positioning device including a frame 122, a femoral positioning plate 124, and a tibial positioning plate 126. The frame 122 is configured to provide support for a patient and position the patient's knee while in the weight bearing position with the femoral positioning plate and tibial positioning plate behind the patient's leg.

The positioning device 120 may include and/or may be used in conjunction with a base 130 and a platform 142 configured to allow the patient 10 to be at a height raised from the ground level. However, various other configurations have been contemplated. Additionally, the frame 122 includes an extending portion 134 which is coupled to and extends upward from the base 130. The extending portion 134 may include one or more rails 128 disposed perpendicular to the base 130 portion. In the example shown, the extending portion 134 includes rails 128 extending adjacent the rear two corners of the base 130 portion.

Moreover, the frame 122 includes a patient engaging portion 136 including one or more handles configured to be engaged by a patient's hand or arm. In the example shown, the patient engaging portion 136 includes two rails 137 (e.g., handles) extending parallel to one another and parallel with the base 130. In the example shown, the handles extend approximately the same length as the base 130, however, various other configurations have been contemplated. The handles are removably or slidably coupled to the extending portion 134 such that the engagement portion can easily accommodate patients of varying heights.

The positioning device 120 also includes the tibial positioning plate 126 and the femoral positioning plates 124 which are similar to the tibial positioning plate 26 and the femoral positioning plates 24 as described above with respect to the positioning device 20. The tibial positioning plate 126 and the femoral positioning plates 124 are fixedly coupled to one another such that the tibial positioning plate 126 and femoral positioning plate 124 are movable as a unit. The femoral positioning plate and tibial positioning plate are sized sufficiently to receive a posterior or back side of a patient's leg.

In accordance with another exemplary embodiment, the subject disclosure provides an x-ray system that includes an x-ray machine 50 configured to output an x-ray beam 52 at an x-ray beam angle. The x-ray machine is configured to output the x-ray beam at an angle of about 0 degrees relative to a vertical axis so as to be able to x-ray the patient's knee while weight bearing. The x-ray system also includes the positioning device 20, 120. The x-ray machine may be configured so as to be positioned above the positioning device.

In operation, the positioning device 20, 120 is configured to provide an unobstructed view of the patellofemoral joint in its functional position, such as when the patient is weight bearing on the patellofemoral joint. The x-ray machine 50 configured to output the x-ray beam 52 is arranged near the patient 10 and the positioning device 20, 120. The patient 10 enters the positioning device 20, 120 at the opening and the patient 10 squats until the femur 12 of the patient 10 (i.e., the patient's thigh) is aligned with the femoral positioning plate 24, 124 and the tibia 14, 114 of the patient 10 (i.e., the patient's lower leg) is aligned with the tibial positioning plate 26, 126. In some examples, the femoral positioning plate 24, 124 and the tibial positioning plate 26, 126 are configured to be engaged with a posterior area of the patient's leg, (see FIG. 7). In other examples, the femoral positioning plate 24, 124 and the tibial positioning plate 26, 126 are configured to engage an anterior area of the patient's leg (see FIGS. 4-6). The positioning device 20, 120 helps position the lower leg (i.e., tibia 14) and thigh (i.e., femur) of the patient 10 such that the patient's knee flexion is approximately 45° and the patient's thigh is at approximately 30° with respect to the x-ray beam (FIG. 3b). Once the patient 10 is positioned on the positioning device 20, 120, the x-ray machine 50 outputs the x-ray beam 52 and obtains the x-ray in a weight bearing merchant view.

The positioning device 20, 120 as described herein allows functional radiographic views of the patellofemoral joint when the extensor mechanism of the knee is active. The positioning device 20, 120 supports the patient 10 in a repeatable position to ensure a 45-degree knee flexion and a 30-degree angle between the patient's femur 12 and the x-ray beam 52 producing images to allow surgeons or other physicians to assess the status of the patellofemoral joint and determine a best medical plan for the patient.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims. This listing of the claims replaces all prior versions, and listings, of the claims in this application.

1. A positioning device for generating merchant views of the knee joint, the positioning device comprising:
a frame configured to receive a patient in a standing or squatting position;
a tibial positioning plate coupled to the frame; and
a femoral positioning plate coupled to the frame and above the tibial positioning plate.

2. The positioning device of claim 1, wherein the tibial positioning plate having a major face at about 15° relative to a longitudinal plane of the positioning device; and the femoral positioning plate having a major face at an angle relative to a plane defined by the tibial positioning plate.

3. The positioning device of claim 2, wherein the femoral positioning plate is disposed at an angle of approximately 45° relative to the plane defined by the tibial positioning plate.

4. The positioning device of claim 2, wherein the femoral positioning plate is disposed at an angle of approximately 30° relative to a longitudinal axis of the positioning device.

5. The positioning device of claim 2, wherein one or more of the tibial positioning plate and the femoral positioning plate are movable relative to one another.

6. The positioning device of claim 2, wherein the tibial positioning plate is fixedly coupled to the frame and the femoral positioning plate is movable relative to the frame.

7. The positioning device of claim 2, wherein the femoral positioning plate is moveable along an axis oriented about 15° with respect to a longitudinal axis of the positioning device.

8. The positioning device of claim 2, wherein the femoral positioning plate is moveable along an axis parallel to a major face of the tibial positioning plate.

9. The positioning device of claim 2, wherein the frame includes one or more handles configured for patient engagement.

10. The positioning device of claim 2, wherein the handles are above the femoral positioning plate.

11. The positioning device of claim 2, wherein the frame includes a pair of rails each having a handle assembly movable along the rail.

12. The positioning device of claim 2, wherein the femoral positioning plate is spaced from the tibial positioning plate.

13. The positioning device of claim 2, wherein the femoral positioning plate is spaced from the tibial positioning plate sufficiently to allow a knee joint therebetween.

14. The positioning device of claim 2, wherein the femoral positioning plate is spaced from the tibial positioning plate sufficiently to allow a knee joint to be positioned anteriorly of the femoral and tibial positioning plates.

15. The positioning device of claim 2, wherein the femoral positioning plate is sized sufficiently to receive a posterior side of a patient's legs.

16. The positioning device of claim 2, further comprising:
an x-ray machine configured to output an x-ray beam at an x-ray beam angle of about 0 degrees relative to a longitudinal axis of the positioning device;
the frame having a longitudinal axis substantially parallel to the x-ray beam angle; and
the femoral positioning plate is disposed at an angle of approximately 30 degrees relative to the longitudinal axis.

17. The x-ray system of claim 16, further comprising a tibial positioning plate coupled to the frame and disposed at an angle of approximately 45 degrees relative to a plane defined by a major face of the femoral positioning plate.

18. The x-ray system of claim 16, wherein one or more of the tibial positioning plate and the femoral positioning plate are movable relative to one another.

19. The x-ray system of claim 16, wherein the femoral positioning plate is above the tibial positioning plate.

20. (canceled)

21. A method of evaluating a patella of a patient in its functional position, the method comprising:
positioning a patient in a standing or squatting position on the positioning device of claim 2; and
obtaining a radiograph of the patient's patella while positioned on the positioning device in a weight bearing position.
